(19) United States
(12) Patent Application Publication
(10) Pub. No.: US 2017/0165197 A1
(43) Pub. Date: Jun. 15, 2017

(54) DERMATOLOGICAL STICK COMPRISING PLANT EXTRACTS, METHODS AND USES THEREOF

(71) Applicants: José António Martinez Souto de Oliveira, VILA DO CONDE (PT); Ana Cristina Palmeira de Oliveira, Peraboa (PT); Rita Manuela Palmeira de Oliveira, COVILHA (PT)

(72) Inventors: Ana Cristina Palmeira de Oliveira, Peraboa (PT); Rita Manuela Palmeira de Oliveira, COVILHA (PT); José António Martinez Souto de Oliveira, VILA DO CONDE (PT)

(21) Appl. No.: 14/964,116

(22) Filed: Dec. 9, 2015

(51) Int. Cl. A61K 9/06; A61K 45/06; A61K 47/44; A61K 47/46; A61K 36/28; A61K 36/53

(52) U.S. Cl. CPC A61K 9/06; A61K 36/28; A61K 36/53; A61K 47/44; A61K 47/46; A61K 45/06

(57) ABSTRACT

The present invention relates to dermatological stick compositions containing natural emollient waxes, oils and as active ingredient plants extract, and to uses thereof, in particular in the treatment dermatological disorders, such as lichen scleroses.

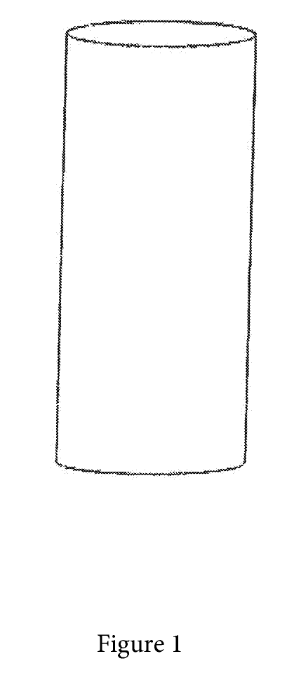

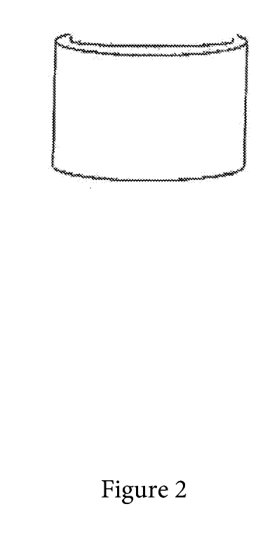

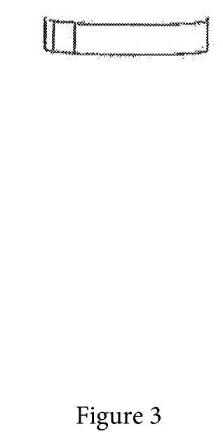

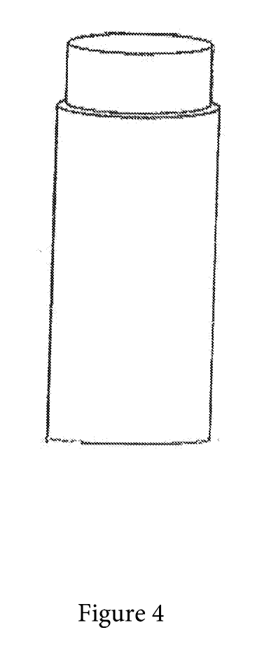

### TECHNICAL FIELD

The present invention relates to dermatological stick compositions containing natural emollient waxes, oils and as active ingredient plants extract, and to uses thereof, in particular in the treatment dermatological disorders, such as lichen scleroses.

The present subject-matter describes a stick composition that may comprise natural emollient waxes and oils and its main effect is to vehicle essential oils and/or other plant extracts with anti-inflammatory activity, antimicrobial, immunomodulatory, anaesthetic, anti-pruritus, and other activities, which are important for the improvement of symptoms and diseases of genital skin, such as lichen scleroses, and other dermatological diseases of inflammatory and autoimmune origin.

### BACKGROUND

The knowledge gathered from the traditional use of plants with medicinal properties has been explored for the selection of therapeutic alternatives for the treatment of diseases for which conventional strategies lack efficacy. Essential oils and other plant extracts have been particularly studied in the pharmaceutical field regarding their antimicrobial bioactivity but also regarding their anti-inflammatory, analgesic, anti-spasmodic and local anaesthetic effects, among others.

The affections that are characterized by high prevalence and recurrence, or that are refractory to currently available therapeutics arise as a preferential work field for the study of the activity of these alternatives. Lichen represents a clear example of a dermatological disease with difficult treatment. Although its aetiology is not known, it is thought to be related with a genetic and autoimmune component, with preferential occurrence in the male and female anogenital region. Lichen is more prevalent in women and affects, more frequently, the labia majora and labia minora, the clitoris, perineum and perianal region, of women of any age but predominantly after the fourth life decade. Although it may occur asymptomatically, lichen frequently causes pruritus and burning, bruise and fissuration, compromising patients' quality of life. High potency topical corticosteroids are the first line therapeutics which may lead to adverse effects regarding epithelial atrophy related to prolonged treatments. Other therapeutic approaches include the topical use (as ointments) of immunomodulatory agents (tacrolimus and pimecrolimus), hormones and retinoids and, in very severe cases, surgical intervention, which have been used with different rates of success and safety profiles. However, there is not a currently available totally effective alternative for lichen treatment and symptoms control. The same is observed for other genital and non-genital dermatological diseases for which it is urgent to find therapeutic alternatives and, mainly, develop formulations that may improve their efficacy. In this context, the development of therapeutic strategies for topical application and local action is particularly interesting since it is associated with higher levels of efficacy (higher concentrations in the site of treatment) and improved safety (due to decreased systemic absorption).

In the context of skin diseases, several plants belonging to the genus Helichrysum spp., Thymus spp., Echinacea spp., chamomile, lavender, have been studied for their therapeutic properties. However, there are no developed products based on natural ingredients, with compositions adapted to damaged skin (lacking irritant ingredients, including synthetic perfumes and preservatives) and designed under the shape of a stick, to vehicle extracts obtained from these plants for the treatment or relief of symptoms associated with these skin diseases.

These facts are disclosed in order to illustrate the technical problem addressed by the present disclosure.

### GENERAL DESCRIPTION

The present disclosure refers to a solid product (medication stick) for skin application and local effect for the treatment or adjunctive treatment of skin diseases or control of symptoms thereof. In an embodiment, the stick composition of the present subject-matter is based on ingredients of natural origin, with no synthetic additives, and vehicles plant extracts and other compounds targeting therapeutic needs previously identified among the pharmaceutical and medical devices markets, namely the need for products with skin regenerating activity, antimicrobial, anti-inflammatory, anti-pruritus and immunomodulatory activity, among others.

The stick composition of the present subject-matter may comprise natural emollient waxes and oils and its main effect is to vehicle essential oils and/or other plant extracts with anti-inflammatory activity, antimicrobial, immunomodulatory, anaesthetic, anti-pruritus, and other activities, which are important for the improvement of symptoms and diseases of genital skin, such as lichen sclerosus, and other dermatological diseases of inflammatory and autoimmune origin.

The stick composition of the present subject-matter is totally different from previously marketed products due to its qualitative composition based on natural products, including mixtures of waxes that were not previously used in the production of these type of pharmaceutical dosage forms. This disclosure was designed to present qualitative and quantitative compositions compatible with genital application which assumes additional innovation since it is a medication stick developed for vulvar application, namely Lichen sclerosus.

Sticks represent a pharmaceutical dosage form rarely used in therapeutics. The Portuguese Pharmacopeia 9.9 defines sticks as “solid preparations for local use, cylindrical or conic in shape, composed by one or several active substances that may be used as presented or after being dissolved or dispersed in a simple or composed excipient which may be soluble or melt at body temperature”.

Lichen sclerosus (LIE-kun skluh-ROW-sus) is an uncommon condition that creates patchy, white skin that's thinner than normal. Lichen sclerosus can affect skin anywhere on your body. But it most often involves skin of the vulva, foreskin of the penis or skin around the anus.

One aspect of the present subject-matter describes a dermatological stick composition for use in the treatment of dermatological diseases of inflammatory or autoimmune origin comprising:

0-15% (w/w) of candelilla wax,

0-15% (w/w) of carnauba wax,

1-50% (w/w) of ceeswax,

0-40% (w/w) of cocoa butter

10-70% (w/w) of castor oil,

0.5-3% (w/w) of plant extracts as active component,

wherein the said extracts are selected from plant extract of a list consisting of: H. italicum, Thymus spp., T. mastichina, T. vulgaris, Echinacea spp., E. purpurea, and mixtures thereof.

In an embodiment the dermatological stick composition of the present subject-matter may further comprise: 0.5-30% (w/w) of petrolatum, a natural antioxidant, 0.5-2% (w/w) of propolis, combinations thereof, among others. In a preferred embodiment the natural antioxidant may be vitamin E.

In an embodiment the dermatological stick composition of the present subject-matter may comprise: 0.5-3% (w/w) of candelilla wax, 0.5-3% (w/w) of carnauba wax, 10-30% (w/w) beeswax, 5-20% (w/w) of cocoa butter, 45-70% (w/w) of castor oil, 0.5-2% (w/w) of plant extracts.

In an embodiment the dermatological stick composition of the present subject-matter may further comprise at least a second active substance selected from the list consisting of: an anaesthetic, an immunomodulator, an antimicrobial agent, or mixtures thereof.

In an embodiment the dermatological stick composition of the present subject-matter may be use in the treatment of dermatological diseases of the genital region, in mammal, in particular in human, more in particular in woman, even more in particular in the vulva, penis or anus.

In an embodiment the dermatological stick composition of the present subject-matter may be use in the treatment of dermatological diseases in the vulva, in particular for use in the treatment of lichen sclerosus.

The stick composition of the present disclosure was tested in several women patients with lichen sclerosus—a dermatitis which affects the vulva, including the large and small lips. All tested patients exhibited significant improvements after daily use of a dermatological stick composition of the present subject-matter for a period of two months.

In an embodiment, the daily form consists of the application of the dermatological stick composition of the present subject-matter once a day during the morning.

### DETAIL DESCRIPTION

The present subject-matter described a stick composition that may be prepared by mixing solid waxes (such as beeswax, carnauba wax, cadelilla wax, cocoa butter, among others) with oils (petrolatum, castor oil, cotton oil or other natural oil) and active agents of natural origin (plant extracts or plant derived products or from other natural source). It is, therefore, a soft stick (in accordance with the definition of the Portuguese Pharmacopoeia) which is hydrophobic. The use of a natural antioxidant agent (preferably vitamin E) is desired to assure the stability of the oils. Depending on the selected ingredients and their mass percentages in the final compositions, sticks with different textures may be obtained and may be adapted to the skin region to be treated. The developed sticks are stable at room temperature. Similar qualitative compositions with minimized weight percentages of high melting point waxes result in semisolid formulations.

Throughout this document, references to % will always refer to “weight by weight”, which is the proportion of a particular substance within the weight of the total composition, as measured by weight or mass.

In an embodiment, the stick composition of the present disclosure may have the following elements:
1. A stick composition comprising candelilla wax (0-15%), carnauba wax (0-15%), beeswax (1-50%), castor oil (10-70%) or other natural oil (preferably of vegetal origin) such as cotton oil or grape seeds oil, cocoa butter (0-40%), active components: essential oil or other plant extract or mixtures of plant extracts with therapeutic interest for the targeted use (0.5-3% each). The plants used for the preparation of the extracts may belong to the genus of: Helichrysum spp. (example H. italicum), Thymus spp. (example T. mastichina, T. vulgaris), Echinacea spp. (example E. purpurea), Chammomile, Lavender, or mixtures thereof, among others.
2. Compositions similar to the previous further comprising petrolatum (0.5-30%).
3. Compositions similar to those described in the previous points further comprising a natural antioxidant (for example Vitamin E) (0.5-2%).
4. Compositions similar to those described in the previous points further comprising other type of natural products (such as propolis).
5. Compositions similar to those described in the previous points further comprising other active component of synthetic origin with therapeutic activity which may improve the therapeutic action of the natural products included.

In an embodiment for improved results, the compositions may do not include preservatives (such as parabens), potentially irritant additives and complex synthetic excipients frequently used in these type of solid preparations.

In an embodiment, the method of preparation of the stick is based on melting the waxes, in a water bath (maximum temperature reached 90° C.), either separately and then mixed or sequentially melted, considering the melting point of each single ingredient, with simultaneous mixing. The plant extracts and other natural products are added only when the emollient base is prepared assuring that their submission to heat in minimized.

In an embodiment, the stick compositions present physical, chemical and microbiological stability verified by textural analysis, dosing of active agents and microbiological quality and stability tests, in accordance with the related official standards.

In an embodiment, the preparation process of the sticks may begin with melting the higher melting point waxes, in a water bath, at the maximum temperature of 90° C. Then the lowest melting point waxes are melted and mixed with the previous, avoiding the inclusion of air. Then the liquid ingredients (oils) are heated with the active components dissolved and they are mixed with the previous mixture, under stirring, before pouring the mass on the moulds of adequate size. To assure a homogenous solidification process it is necessary to add an excess of melted mass, avoiding the occurrence of a central hole as a result of the contraction of the waxes during the solidification process. Once the solidification process is concluded, the stick is removed from the moulds and packed in the final packages.

In an embodiment, the prepared stick is dispensed in plastic packages, preferably with opaque cover. The packages are preferably cylindrical in shape, with a rotatory mechanism in the base and a platform where the recently prepared stick is fitted becoming protected from light and air. The use of a rotatory mechanism in the base allows for the exposition of the surface of the stick to skin contact avoiding the contact with the package.

In an embodiment after application, the device allows the correct packaging of the stick inside the cylindrical tube, covered with the cover until the next use. The packaging shall be opaque to avoid the degradation of the formulation, due to the effect of light.

In an embodiment, the developed formulations are tested in the development process and quality control process regarding their physical characteristics, particularly concerning textural and hardness determination of formulations, chemical characteristics, such as oil resistance to oxidation and dosing of the active ingredients and microbiological quality or resistance to contamination after challenge in accordance with the microbiological tests inscribed in the Portuguese Pharmacopeia.

The evaluation of these parameters throughout time assures the stability of the products and the definition of expiration dates.

In an embodiment, the stick composition of the present disclosure is used for the treatment or improvement of dermatological symptoms, namely inflammation and pruritus. It was particularly designed for application in genital skin of mammals, namely in genital skin female mammal, in particular the vulva (namely human vulva), particularly in the treatment of lichen sclerosus.

In an embodiment, the stick composition of the present disclosure can be used in the treatment of other genital and non-genital dermatological diseases that benefit from local application, in reduced areas, of products of natural origin, such us other dermatosis and allergic dermatological diseases.

Although the dermatological use of medication sticks is described, there are no known formulations designed for the genital skin, in particular for the vulva, with composition similar to those presented in this disclosure.

Throughout the description and claims the word “comprise”, “comprising” and variations of this word, have no intention of excluding other technical characteristics, additives, components, or steps. Additional objects, advantages and characteristics of the disclosure will become evident for the experts in the technique after examination of the description or may be learnt from the practice of the disclosure. Furthermore, the present disclosure covers all possible combinations of particular or combination embodiments herein described.

The disclosure should not be seen in any way restricted to the embodiments described and a person with ordinary skill in the art will foresee many possibilities to modifications thereof.

The above described embodiments are combinable. The following claims further set out particular embodiments of the disclosure.

1. A dermatological stick composition for use in the treatment of dermatological diseases, in particular dermatological diseases with inflammatory or autoimmune origin, comprising:
0-15% (w/w) of candelilla wax,
0-15% (w/w) of carnauba wax,
1-50% (w/w) of beeswax,
0-40% (w/w) of cocoa butter
10-70% (w/w) of castor oil,
0.5-3% (w/w) of plant extracts as active component,
wherein the said extracts are selected from plant extract of a list consisting of: H. italicum, Thymus spp., T. mastichina, T. vulgaris, Echinacea spp., E. purpurea, and mixtures thereof.

2. The dermatological stick composition according to claim 1 further comprising: 0.5-30% (w/w) of petrolatum, or a natural antioxidant, or 0.5-2% (w/w) of propolis, or combinations thereof.

3. The dermatological stick composition according to claim 1 wherein the natural antioxidant is vitamin E.

4. The dermatological stick composition according to claim 1, comprising:
0.5-3% (w/w) of candelilla wax,
0.5-3% (w/w) of carnauba wax,
10-30% (w/w) of beeswax,
5-20% (w/w) of cocoa butter,
45-70% (w/w) of castor oil, and
0.5-2% (w/w) of plant extracts.

5. The dermatological stick composition of claim 1, further comprising at least a second active substance selected from the group consisting of: an anaesthetic, an immunomodulator, an antimicrobial agent, and mixtures thereof.

6. The dermatological stick composition of claim 1, for use in the treatment of dermatological diseases.

7. The dermatological stick composition of claim 1, for use in the treatment of the genital region.

8. The dermatological stick composition of claim 1, for use in the treatment of dermatological diseases in the vulva, penis or anus.

9. The dermatological stick composition of claim 1, for use in the treatment of lichen sclerosus.
